(19) United States
(12) Patent Application Publication
(10) Pub. No.: US 2015/0231093 A1
(43) Pub. Date: Aug. 20, 2015

(54) METHODS AND COMPOSITIONS FOR COUNTERMEASURES AGAINST RADIATION

(71) Applicant: THE UNIVERSITY OF CHICAGO, Chicago, IL (US)

(72) Inventors: Richard C. Miller, Wheaton, IL (US); Jeffrey S. Murley, Woodridge, IL (US); David J. Grdina, Naperville, IL (US)

(21) Appl. No.: 14/624,157

(22) Filed: Feb. 17, 2015

Related U.S. Application Data
(60) Provisional application No. 61/940,773, filed on Feb. 17, 2014.
(60) Provisional application No. 61/975,438, filed on Apr. 4, 2014.

(51) Int. Cl. A61K 31/155; A61K 31/661; A61K 31/145; A61K 45/06

(52) U.S. Cl. CPC A61K 31/155; A61K 45/06; A61K 31/661; A61K 31/145

(57) ABSTRACT

Described are methods and compositions for treating subjects for symptoms from unplanned radiation or protecting subjects therefrom. Embodiments include methods of treating subjects exposed to unplanned radiation with pharmaceutical compositions comprising a biguanide compound. In further embodiments, methods and compositions are provided for radioprotection by administering pharmaceutical compositions comprising a biguanide compound and additional therapeutic compounds.

### CROSS-REFERENCE TO RELATED APPLICATIONS

This application claims priority to U.S. Provisional Application No. 61/940,773 filed Feb. 17, 2014, and U.S. Provisional Application No. 61/975,438 filed Apr. 4, 2014, which are hereby incorporated by reference in their entireties.

The invention was made with government support under Grant No. R01-CA132998 awarded by the National Institutes of Health and Grant No. DE-SC0001271 awarded by the Department of Energy. The government has certain rights in the invention.

### BACKGROUND OF THE INVENTION

1. Field of the Invention

The present invention relates generally to the field of biology and medicine. More particularly, it concerns methods and compositions for radiation protection and/or radiation toxicity mitigation using chemical compounds.

2. Description of Related Art

Following the horrific events of Sep. 11, 2001, there has been a concerted effort to protect the population from radiological terrorism. A major focus of this effort is in the development of chemical agents that can protect against the toxic effects of ionizing radiation. As a result of the 2004 report of an National Cancer Institute Workshop (Stone, et al., 2004.), the development of radioprotective agents was subdivided into three categories: prophylactic agents that protect if administered prior to radiation exposure; mitigator agents that are administered during or following irradiation that can prevent or lessen radiation toxicity; and therapeutic agents that are administered following irradiation to treat and enhance recovery from radiation induced damage. While these terms represent three distinct classes of radioprotectors, it is possible that some radioprotective agents can exert protective effects across all three of these artificial categories (Coleman, et al., 2004; Weiss & Landauer, 2009). At present there is only one prophylactic radioprotector that has been approved by the U.S. Food and Drug Administration (FDA) and that is amifostine for the protection against xerostomia induced by radiation exposure in the treatment of head and neck cancer (Grdina, et al., 2000).

There remains a need to develop an effective radiation countermeasure agent that can be administered after radiation exposure to help many potential victims of a radiological accident or terrorist attack, because immediate aid could be delayed due to the resulting chaos and confusion that would exist.

### SUMMARY OF THE INVENTION

Certain embodiments are based on, in part, on the data showing that metformin, alone or with other therapeutic compounds, can protect animals from exposure of unplanned radiation even when administered hours after the radiation exposure, for example, after more than 24 hours following radiation exposure.

Because this protection can save animals from accidental radiation, acute radiation symptoms, or even radiation-induced death, this protection is different from classical pre-irradiation administration and protection against the long-term effects of planned therapeutic radiation, which effects may include down-regulation of immediate radiation induced-oxidative stress or protection against endogenous reactive oxygen species and associated DNA damage.

In further embodiments, compositions and methods are based on, in part, on the surprising determination that the co-administration of biguanide drugs such as metformin and additional therapeutic drugs after radiation exposure can synergistically treat radiation-induced damage at similar efficacy as administering radio-protecting agents before radiation exposure. This provides additional advantages for radiation protection after radiation exposure.

Certain embodiments include a method of treating a subject exposed to radiation, particularly a near lethal level or a lethal level of radiation. The method may comprise administering to the subject an effective amount of a pharmaceutical composition comprising therapeutic compounds that protect the subject after the radiation. In a particular example, the therapeutic compounds is a biguanide compound.

As contemplated in certain aspects, one non-limiting advantage of the present methods and compositions is to treat for unplanned radiation, especially a high level of radiation. Thus, in some embodiments, the subject has not been subject to any radiation protection, including administering an effective amount of a radio-protective pharmaceutical composition comprising a biguanide compound before being exposed to radiation, or particularly a near lethal level or a lethal level of radiation. In some aspects, the methods may include administering the pharmaceutical compositions only after the subject has been exposed to radiation, particularly a lethal level of radiation or a radiation suspected of being at a lethal level.

In certain aspects, the subject may be administered a pharmaceutical composition described herein prior to being exposed to being expected to be exposed to radiation for a radioprotective effect. In further aspects, the subject may be administered a pharmaceutical composition both prior or after being exposed to radiation.

In further aspects, the subject was exposed in a lethal level of radiation or a radiation suspected of being at a lethal level. In certain aspects, the subject was determined to exhibit the symptoms of acute radiation syndrome or radiation-induced cytotoxicity.

The therapeutic compounds can be provided at a dose of at least about, at most about, or about 0.001, 0.1, 0.5, 1, 2, 3, 4, 5, 6, 7, 8, 9, 10, 25, 50, 100, 200, 400, 500, 1000, 2000, 5000 mM, mg/kg, or mg/kg/day, including all values or ranges there between. In particular embodiments, the therapeutic compounds such as a biguanide compound is administered at a dosage of 1 to 500 mg/kg weight. In certain aspects a therapeutic compound is administered at a dose of at least about, at most about, or about 20 mg/kg to about 500 mg/kg, and more particularly about 75-250 mg/kg or mg/kg/day. The therapeutic compound can be administered 1, 2, 3, 4, 5, or more times a day, a week, a month, or a year. In certain aspects, a therapeutic compound is administered about every 24, 48, 72, 96, 120 hours or any range derivable therein. The therapeutic compound may be provided intravenously, i.v., or orally, but also methods may include administration via other enteral routes—intra-arterial, subcutaneous, intraperitoneal injection, infusion or perfusion—or via inhalation routes.

In some embodiments, the therapeutic compound is a biguanide compound such as metformin, phenformin, or buformin. In some embodiments, the pharmaceutical composition further comprises a second drug, or wherein the method further comprises administering a separate pharmaceutical composition comprising a second drug. In further embodiments, the second drug is phosphorothioate compound or an associated metabolite, a sulfhydryl compound, or a prodrug or salt thereof.

In some embodiments, the second drug is a phosphorothioate compound or an associated metabolite, or a prodrug or salt thereof For example, the therapeutic compound or second drug is phosphorothioate compound or an associated metabolite. Phosphorothioates or an associated metabolite used in certain aspects of the invention are exemplified by, but not limited to S-2-(3-aminopropylamino)ethyl phosphorothioic acid (amifostine, WR-2721), 2-[(aminopropyl)amino] ethanethiol (WR-1065), S-1-(amino ethyl) phosphorothioc acid (WR-638), 5-[2-(3-methylaminopropyl)aminoethyl] phosphorothioate acid (WR-3689), S-2-(4-aminobutylamino) ethylphosphorothioic acid (WR-2822), 3-[(2-mercapto ethyl)amino] propionamide p-toluenesulfonate (WR-2529), S-1-(2-hydroxy-3-amino) propyl phosphorothioic acid (WR-77913), 2-[3-(methylamino) propylamino] ethanethiol (WR-255591), S-2-(5-aminopentylamino) ethyl phosphorothioic acid (WR-2823), [2-[(aminopropyl) amino] ethanethiol] N,N,′ -dithiodi-2,1-(ethanediyl) bis-1,3 -propanediamine (WR-33278),1-[3-(3-aminopropyl) thiazolidin-2-Y1]-D-gluco-1,2,3,4,5 pentane-pentol dihydrochloride (WR-255709), 3-(3 -methylaminopropylamino) prop anethiol dihydrochloride (WR-151326), S-3-(3-methylaminopropylamino) propyl phosphorothioic acid (WR-151327), a prodrug or salt thereof In particular aspects, the phosphorothioate is WR-2721.

In further embodiments, the second drug is a sulfhydryl compound, or a prodrug or salt thereof The sulfhydryl compound may be any compounds that have a sulfhydryl group. In some embodiments, the therapeutic compound or second drug is a sulfhydryl compound selected from the group consisting of an aminothiol compound, an angiotensin converting enzyme inhibitor, a detoxifying agent, an anti-mucolytic agent, and a combination thereof

In some embodiments, the aminothiol compound is 2-[(aminopropyl) amino] ethanethiol (i.e., WR-1065) or a prodrug or salt thereof In further embodiments, the angiotensin converting enzyme inhibitor is captopril, zofenopril, fosinopril or enalapril, or a prodrug or salt thereof. In some embodiments, the detoxifying agent is MESNA (sodium 2-sulfanylethanesulfonate) or a prodrug or salt thereof In some embodiments, the anti-mucolytic agent is a modified form of cysteine such as N-acetyl-cysteine (NAC) or a prodrug or salt thereof.

In further embodiments, the pharmaceutical composition is administered subcutaneously, intravenously, topically, transdermally, by inhalation, or orally. In some embodiments, the pharmaceutical composition is administered subcutaneously. In some embodiments, the pharmaceutical composition is administered orally. In further embodiments, there may be provided separate pharmaceutical compositions for different routes of administration.

For topical or transdermal administration, the composition may be administered in any suitable format, such as a lotion, salve, gel, cream, balsam, tincture, cataplasm, elixir, paste, spray, collyrium, drops, suspension, dispersion, hydrogel, film, ointment, emulsion or powder; or alternatively, the composition may be administered using a transdermal patch, a bandage, a gauze, a wound or sore dressing, or adhesive tape.

In some embodiments, the pharmaceutical composition is administered immediately after or only immediately after the subject is first exposed to radiation. In some embodiments, the pharmaceutical composition is administered at least about 4 hours after the subject is first exposed to radiation. In some embodiments, the pharmaceutical composition is administered at least about 6 hours after the subject is first exposed to radiation. In some embodiments, the pharmaceutical composition is administered at least about 18 hours after the subject is first exposed to radiation. In some embodiments, the pharmaceutical composition is administered at least about 24 hours after the subject is first exposed to radiation. In some embodiments, the pharmaceutical composition is administered at least about 36 hours after the subject is first exposed to radiation. In some embodiments, the pharmaceutical composition is administered at least about 48 hours after the subject is first exposed to radiation.

In further embodiments, the timing of administration is relevant. One, 2, 3, 4, or 5 doses may be administered to the subject after the exposure to radiation, and in some embodiments, the dose is administered only after that exposure to radiation. In some embodiments, the dose of pharmaceutical compositions or therapeutic compounds are administered at least, about, more than, or at most 1, 2, 3, 4, 5, 6, 7, 8, 9, 10, 11, 12, 13, 14, 15, 16, 17, 18, 20, 21, 22, 23, 23, 24, 25, 26, 27, 28, 29, 30, 31, 32, 33, 34, 35, 36, 37, 38, 39, 40, 41, 42, 43, 44, 45, 46, 47, or 48 hours (or any range derivable therein) after or only after radiation exposure.

Some embodiments include a human subject. In some embodiments, the subject is a non-human animal, such as a mouse, a dog, a cat, a pig, a horse, a cow, or any mammal. In some embodiments, the pharmaceutical composition is administered to the subject after the subject is tested for the level of radiation exposure or determined for the occurrence of radiation exposure.

Some embodiments include monitoring the subject for radiation-induced damage. In some embodiments, the subject is monitored for radiation-induced cell death. Further embodiments comprise of administering multiple doses of the effective amount of the pharmaceutical composition comprising a biguanide compound. In some embodiments, the multiple doses are administered at a time interval(s) of 0.1, 0.5, 1, 2, 3, 4, 5, 6, 7, 8, 9, 10, 11, 12, 13, 14, 15, 16, 17, 18, 20, 21, 22, 23, 23, or 24 hours (or any range derivable therein).

Some embodiments include a method of treating a subject exposed to radiation, comprising administering to the subject an effective amount of a pharmaceutical composition comprising a biguanide compound after the subject has been exposed to radiation, wherein the subject has not been administered any biguanide compound for radiation protection before being exposed to radiation.

Certain embodiments include a method of treating a subject determined to have acute radiation syndrome or radiation-induced cytotoxicity, comprising administering to the subject an effective amount of a pharmaceutical composition comprising a biguanide compound after the subject has been determined to have acute radiation syndrome or radiation-induced cytotoxicity.

Certain embodiments include a method of treating a subject exposed to a radiological incident, comprising administering to the subject an effective amount of a pharmaceutical composition comprising a biguanide compound. In particular aspects, the radiological incident comprises a near lethal or lethal level of radiation or a radiation suspected of being at a lethal level.

Some embodiments include a method of treating a subject exposed to a radiological incident, comprising administering to the subject an effective amount of a pharmaceutical composition comprising metformin at least, about, more than, or at most 1, 2, 3, 4, 5, 6, 7, 8, 9, 10, 11, 12, 13, 14, 15, 16, 17, 18, 20, 21, 22, 23, 23, 24, 25, 26, 27, 28, 29, 30, 31, 32, 33, 34, 35, 36, 37, 38, 39, 40, 41, 42, 43, 44, 45, 46, 47, or 48 hours (or any range derivable therein) after the subject is first exposed to the radiological incident.

Some embodiments include a method of treating a subject exposed to a radiological incident or radiation, comprising administering to the subject an effective amount of a pharmaceutical composition comprising a biguanide compound and a phosphorothioate compound or an associated metabolite, a sulfhydryl compound or a prodrug or salt thereof. In some embodiments, the biguanide compound is metformin or a prodrug or salt thereof; and the sulfhydryl compound is WR1065, NAC, captopril or MESNA or a prodrug or salt thereof.

In certain embodiments, there are methods of treating a subject exposed to whole body radiation comprising administering to the subject only after the exposure a composition comprising a biguanide compound and/or a phosphorothioate compound. In certain embodiments, there are methods of treating a subject exposed to a sublethal or lethal level of whole body radiation comprising administering to the subject a composition comprising a metformin and/or an amifostine only after the subject has been exposed.

In certain aspects, the radiation is an ionizing radiation, such as radiation from Gamma rays, X-rays, and the higher ultraviolet part of the electromagnetic spectrum. In further aspects, the radiation is a near lethal or a lethal level of radiation, or a radiation suspected of being at a lethal level.

In other methods, steps may include one or more of the following steps: ordering multiple doses, compositions or devices comprising a pharmaceutical composition containing a biguanide compound such as metformin and/or a phosphorothioate compound, such as amifostine; storing or stockpiling the multiple doses, compositions, or devices; preparing multiple doses for long-term storage, where long-term storage means at least 1 year, but can be 3, 4, 5, 6, 7, 8, 9, 10 or more years; providing or distributing multiple doses at a single location (meaning within a 5, 6, 7, 8, 9, 10 or more mile radius—or any range derivable therein) within a limited time span (meaning a time within at least 1 month, or 1, 2, 3 or 4 weeks, or 1, 2, 3, 4, 5, 6, or 7 days, or within 24 hours); or administering multiple doses, compositions or devices to multiple people. In some embodiments, the multiple doses is or is at least 100, 200, 300, 400, 500, 600, 700, 800, 900, 1000, 2000, 3000, 4000, 5000 or more (or any range derivable therein). In further embodiments, the number of subjects is or is at least 100, 200, 300, 400, 500, 600, 700, 800, 900, 1000, 2000, 3000, 4000, 5000 or more (or any range derivable therein). The doses and or subjects may be administered or treated within a limited time span according to some embodiments. It is further contemplated that long-term storage may refer to storage that does not require storage at or below 4 degrees Celsius, according to some embodiments. It is also specifically contemplated that one or more steps may be implemented by government workers, military personnel, or a combination thereof.

Certain embodiments include a kit or apparatus or medical device comprising one or more doses of: a first pharmaceutical composition comprising metformin or a prodrug or salt thereof; and a second pharmaceutical composition comprising a phosphorothioate compound or an associated metabolite, i.e., a metabolite thereof, a sulfhydryl compound or a prodrug or salt thereof. The sulfhydryl compound may be selected from the group consisting of WR1065, NAC, captopril, MESNA, or a prodrug or salt thereof, or may be WR1065, NAC, captopril, MESNA, or a prodrug or salt thereof. In particular aspects, the first and second pharmaceutical composition are individually packaged. In some embodiments, the first or second pharmaceutical composition is in the form of tablets. In further embodiments, the first or second pharmaceutical composition is in the form of syringes.

Further embodiments include a kit comprising one or more doses of a pharmaceutical composition comprising metformin or a prodrug or salt thereof and amifostine, WR-1065, NAC, captopril, MESNA, or a prodrug or salt thereof In further embodiments, there is a composition containing at least both a biguanide and a phosphorothioate (or a salt or prodrug thereof). In some embodiments, the composition may be included in a delivery device. In other embodiments, there is a delivery device comprising separate compositions in which one composition comprises a biguanide compound and another composition comprises a phosphorothioate compound (or salt or prodrug thereof).

In certain aspects, any kit described herein may be further defined as a transdermal patch, a bandage, a gauze, a wound or sore dressing, or adhesive tape. In further aspects, any kit described herein may be further defined as a lotion, salve, gel, cream, balsam, tincture, cataplasm, elixir, paste, spray, collyrium, drops, suspension, dispersion, hydrogel, ointment, emulsion or powder.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—The toxicity profile of metformin (Met) on SA-NH murine sarcoma cells in the dose range of 0.5 to 20 mM following a one hour exposure. Each experiment was performed three times and error bars represent the standard error of the mean (SEM).

FIG. 2—A time course of the radio-protective effects of metformin (Met) on SA-NH murine sarcoma cells at a concentration of 5 mM and an exposure time of one hour administered from 1 to 48 h following irradiation with 4 Gy. Amifostine's active free thiol form WR1065 (WR) was administered at a concentration of 4 mM 30 mM prior to irradiation to serve as a positive control for radioprotective comparison. Each experiment was repeated three times and error bars represent the SEM. P values comparing cell survival at 4 Gy only to those following treatment with Met or WR are presented for comparison.

FIG. 3—A time course of the radio-protective effects of Met on human microvascular endothelial cells (HMEC) at a concentration of 5 mM and an exposure time of one hour administered from 1 to 48 h following irradiation with 4 Gy. WR was administered at a concentration of 4 mM 30 mM prior to irradiation to serve as a positive control for radioprotective comparison. Each experiment was repeated three times and error bars represent the SEM. P values comparing cell survival at 4 Gy only to those following treatment with Met or WR are presented for comparison.

FIG. 4—A time course of the radio-protective effects of Met on mouse embryo fibroblasts (MEF) at a concentration of 5 mM and an exposure time of one hour administered from 1 to 48 h following irradiation with 4 Gy. WR was administered at a concentration of 4 mM either 30 min before irradiation as a positive control for radioprotective comparison or in combination with Met 1 hour or 24 hours following irradiation. Each experiment was repeated three times and error bars represent the SEM. P values comparing cell survival levels exposed to 4 Gy only with those exposed to Met and WR alone or in combination are presented for comparison.

FIG. 5—A comparison of the radio-protective effects of Met at a dose of 250 mg/kg alone or in combination with amifostine (Ami) 400 mg/kg, captopril (Cap) 200 mg/kg, MESNA 300 mg/kg, and N-acetyl-cysteine (NAC) 400 mg/kg administered 24 h following a 7 Gy dose of whole body ionizing radiation using a C3H mouse model and the endogenous spleen colony assay are presented. Ami alone, 400 mg/kg, administered 30 min prior to or 24 h following 7 Gy irradiation are included as positive controls. The number of colonies growing on the surface of spleens from mice in each experimental group 13 days following radiation exposure was counted and serves as a measure of the relative radio-protectiveness of the various experimental treatments. Error bars represent the SEM. P values comparing the number of spleen colonies following the various treatment groups with that following 7 Gy only are presented for comparison.

### DESCRIPTION OF ILLUSTRATIVE EMBODIMENTS

Countermeasures against unplanned irradiation include a wide range of potential molecular and cellular interventions. Here in some embodiments, methods and compositions are provided for using therapeutic compounds to treat subjects exposed to unplanned irradiation. They may offer advantages including protection from acute radiation damage, even when the therapy is administered hours after the radiation exposure. These methods and compositions may protect subjects from high level of radiation, such as lethal levels or near lethal levels.

It is contemplated that such protection may be different from protection from low level of radiation, such as used in diagnostic radiology methods including CT scans and other low radiation exposure methods (for example, ≦500 mille Sevierts). The latter protection is through reduction of radiation-induced DNA damage to thereby minimize mutagenesis and carcinogenesis. The low level of radiation leads to gross but non-lethal chromosomal and induces genomic instability characterized by hyper recombination.

Contrary to preventing damage due to a low dose of therapeutic radiation, compositions and methods in certain embodiments may be particularly suited for subjects exposed to unplanned radiation exposures, especially subjects exposed to a high level of radiation that is not for therapeutic purposes.

### I. Radiation

In certain embodiments, methods are provided to treat subjects exposed to incidental radiation, such as a disaster involving nuclear release or radiation like a nuclear explosion. The subject may have been exposed or is expected to be exposed or suspected of having been exposed to radiological incidents, which include all nuclear incidents like radiation exposure from nuclear reactor accidents and exposure to radioactive materials (e.g., Cs 137, cobalt 60, iodine 131) accidently or in dirty bombs and other terrorist devices. For example, nuclear incidents include fission or fusion nuclear reactions such as would be seen in and atomic bomb or a nuclear reactor exploding. It is specifically contemplated that sun exposure and/or UV irradiation is excluded as radiation in some embodiments provided herein.

In certain aspects, the radiation may be ionizing radiation. Examples of ionizing subatomic particles from radioactivity for producing ionizing radiation include alpha particles, beta particles and neutrons. Almost all products of radioactive decay are ionizing because the energy of radioactive decay is typically far higher than that required to ionize. Other subatomic ionizing particles which occur naturally are muons, mesons, positrons, neutrons and other particles that constitute the secondary cosmic rays that are produced after primary cosmic rays interact with Earth's atmosphere. Cosmic rays may also produce radioisotopes on Earth (for example, carbon-14), which in turn decay and produce ionizing radiation. Cosmic rays and the decay of radioactive isotopes are the primary sources of natural ionizing radiation on Earth referred to as background radiation.

In space, natural thermal radiation emissions from matter at extremely high temperatures (e.g. plasma discharge or the corona of the Sun) may be ionizing. Ionizing radiation may be produced naturally by the acceleration of charged particles by natural electromagnetic fields (e.g. lightning), although this is rare on Earth. Natural supernova explosions in space produce a great deal of ionizing radiation near the explosion, which can be seen by its effects in the glowing nebulae associated with them.

Ionizing radiation can also be generated artificially using X-ray tubes, particle accelerators, and any of the various methods that produce radioisotopes artificially.

Ionizing radiation may be invisible and may not be directly detectable by human senses, so radiation detection instruments such as Geiger counters may be required. However, ionizing radiation may lead to secondary emission of visible light upon interaction with matter, such as in Cherenkov radiation and radioluminescence.

Ionizing radiation is applied constructively in a wide variety of fields such as medicine, research, manufacturing, construction, and many other areas, but presents a health hazard if proper measures against undesired exposure are not followed. Exposure to ionizing radiation can cause damage to living tissue, and can result in mutation, radiation sickness, cancer, and death.

In particular embodiments, the subject has been exposed to a high level of radiation. The high level can include any levels higher than any level used in currently therapeutic radiation, such as radiation therapy for cancer.

The high level may include any lethal level or near lethal level of radiation. For example, the mean lethal dose of radiation required to kill 50% of humans 60 days after whole-body irradiation (LD50/60) is between 3.25 and 4 Gy without supportive care, and 6-7 Gy when antibiotics and transfusion support are provided.

A lethal dose (LD) is an indication of the lethality of a given substance or type of radiation. Because resistance varies from one individual to another, the ‘lethal dose’ represents a dose (usually recorded as dose per kilogram of subject body weight) at which a given percentage of subjects will die. The LD may be based on the standard person concept, a theoretical individual that has perfectly “normal” characteristics, and thus not apply to all sub-populations.

Lethal doses may be expressed as median lethal dose (LD50), the point where 50% of test subjects exposed would die, in the units of mg/kg body weight. LD values for humans may be estimated by extrapolating results from human cell cultures.

Lethal dose of whole body radiation may be defined as a lethal dose to kill 50% of the population such as: a) bone marrow or hematopoietic death 2.5 to 5 Gy; b) gastrointestional syndrome 5 to 12 Gy. This is for external radiation administered acutely (Radiobiology for the Radiologist, Eric J. Hall and Amato J. Giaccia Editors, Seventh Edition, Lippincott Williams and Wilkins, a Wolters Kluwer business, Philadelphia, Pa., 2012, Chapter 8, pg 114). This may be a typical scenario for a dirty bomb, a radiological terror device, a nuclear accident or explosion. In other aspects, a lethal level of radiation may include at least or more than 50, 500 mSv, 1, 2, 3, 4, 5, 6, 7, 8, 9, 10, 15, 20, 30, 40, 50, 100 Sv or any range or number derived therefrom. In particular aspects, the lethal level of radiation may be at least 500 mSv.

In some aspects, the lethal level of radiation may be a level sufficient to cause gastrointestional syndrome. In further aspects, the lethal level of radiation may be a level sufficient to cause bone marrow or hematopoietic death because transplantation may be used to save the subject.

In further embodiments, the subjects to be treated have not been exposed to radiation therapy for a disease, such as cancer. In other embodiments, the subjects to be treated have been exposed to radiation therapy but also have been exposed to radiological incidents or unplanned radiation before administering any compositions and treating any methods described herein.

In other embodiments, the subjects to be treated have been exposed to radiation that is non-therapeutic radiation. “Non-therapeutic radiation,” as described herein, refers to radiation that is not desired or intended for diagnosis and/or therapy. The non-therapeutic radiation thus does not include medical procedures involving radiation such as diagnostic radiology CT scans or radiation therapy of cancer. Medical radiation is usually applied to a subject at low doses, such as less than 500 mille Sieverets or less (50 rads for gamma rays) that one needs to protect against cancer induction, and may be in fractions. These are not lethal doses.

Examples for radiation doses can help illustrate relative magnitudes. These are meant to be examples only, not a comprehensive list of possible radiation doses. An “acute dose” is one that occurs over a short and finite period of time, while a “chronic dose” is a dose that continues for an extended period of time so that it is better described by a dose rate.

A sievert (Sv) is a unit of effective dose of radiation. A Sievert can be calculated by multiplying Gy multiplied with weighting factor specific to each type of radiation and organ

Examples of non-lethal level of radiation or therapeutic radiation may include 10 to 30 mSv for single full-body CT scan. A person's radiation exposure due to all natural sources amounts on average to about 2.4 millisievert (mSv) per year.

Examples of near lethal or lethal level of radiation may include:

50 mSv: The U.S. 10 C.F.R. § 20.1201(a)(1)(i) occupational dose limit, total effective dose equivalent, per annum;

68 mSv: estimated maximum dose to evacuees who lived closest to the Fukushima I nuclear accidents;

0.50 Sv: The U.S. 10 C.F.R. §20.1201(a)(2)(ii) occupational dose limit, shallow-dose equivalent to skin, per annum;

0.67 Sv: highest dose received by a worker responding to the Fukushima emergency;

4.5 to 6 Sv: fatal acute doses during Goiania accident;

5.1 Sv: fatal acute dose to Harry Daghlian in 1945 criticality accident;

21 Sv: fatal acute dose to Louis Slotin in 1946 criticality accident.

In certain embodiments, the subjects to be treated have been exposed whole-body radiation. The whole-body radiation is a radiation of at least 50, 60, 70, 80, 90, 95, 100% or any range derivable therein of the body. In some embodiments, the whole body radiation may further include receiving more than 50, 100, 200, 500, 1000, 2000, 5000, 10³, 10⁴, 10⁵, 10⁶ mSv or mSv/year or any range derivable therein. In certain embodiments, a subject is administered a composition comprising biguanide and/or a phosphorothioate only after being exposed to whole-body radiation. It is specifically contemplated that whole body radiation is not therapeutic radiation in some embodiments provided herein.

The subject may also be exposed to localized radiation that is only localized to a region of the body.

In further embodiments, methods and compositions may be provided to subjects previously exposed to radiation from terror attacks. The events of 11 Sep. 2001 prompted assessments of vulnerability to many types of terrorism scenarios, amongst which is a collection described as radiological terrorism. An example is the so-called “dirty bomb” involving dispersal of some form a radioactivity with conventional explosive.

Humans and animals are highly susceptible to radiation-induced damage resulting in cellular, tissue, organ and systemic injuries. In accidental radiation exposure, such as a nuclear explosion or a disaster scenario, many victims will suffer from acute radiation syndrome (ARS) to varying degrees. The immediate objectives at a radiation disaster scene are quite different from the radiation treatment of cancer. In such a disaster scenario, early efforts would involve reaching as many afflicted individuals as possible with a treatment that could prolong life, so that victims could be successfully triaged and receive subsequent, in-depth medical care as dictated by their individual condition and afflictions.

Another aspect of such an accidental, or intentional, radiation disaster is that any life-saving drugs or treatments would have to be active at protracted time points following the radiation disaster. This requirement is due to the time it would take to mobilize medical staff, drugs/treatments, and equipment to a disaster scene, so that life-saving drugs/treatments could be administered to victims in need.

The mortality is largely attributed to the haematopoietic syndrome, a consequence of hypoplasia or aplasia of the bone marrow. Cytopenias develop as a result of radiation-induced and normal attrition of mature functional cells, combined with the failure of replacement because of radiation-induced depletion of haematopoietic stem cells and progenitors. The time and extent of cytopenia generally correlate with radiation dose and prognosis, but the kinetics of depletion and recovery of blood cells also varies between the erythropoiesis, myelopoiesis and thrombopoiesis lineages, thrombopoiesis being the slowest. The gastrointestinal syndrome results from ablation of stem cells in intestinal crypts, which in turn leads to denudation of the intestinal mucosa. This injury occurs after whole-body doses in the range of 3-15 Gy and in rodents doses at the upper end of this range usually result in death within about 1 week after irradiation.

In certain embodiments, the subjects to be treated are at risk, suspected of having or determined to have acute radiation syndrome (ARS) (sometimes known as radiation toxicity or radiation sickness). ARS is an acute illness caused by irradiation of the entire body (or most of the body) by a high dose of penetrating radiation in a very short period of time (usually a matter of minutes). The major cause of this syndrome is depletion of immature parenchymal stem cells in specific tissues. Examples of people who suffered from ARS are the survivors of the Hiroshima and Nagasaki atomic bombs, the firefighters that first responded after the Chernobyl Nuclear Power Plant event in 1986, and some unintentional exposures to sterilization irradiators.

For Acute Radiation Syndrome (ARS), the radiation dose must be large, for example, greater than 2 Gy for bone marrow or hematopoietic death, or greater than 5 for gastrointestinal syndrome. Mild symptoms may be observed with doses as low as 0.3 Gy or 30 rads. The dose usually must be external (i.e., the source of radiation is outside of the patient's body). Radioactive materials deposited inside the body have produced some ARS effects only in extremely rare cases. The radiation for ARS usually must be penetrating (i.e., able to reach the internal organs). High energy X-rays, gamma rays, accelerated high energy electrons and charged particles, and neutrons are penetrating radiations. The radiation dose may have been delivered in a short time (usually a matter of minutes) or a longer time in terms of hours.

For ARS, the entire body (or a significant portion of it) usually must have received the dose. Most radiation injuries are local, frequently involving the hands, and these local injuries seldom cause classical signs of ARS.

In particular embodiments, the subjects have been previously exposed to whole body radiation, which is radiation received by the entire body, or a significant portion, such as at least 50, 60, 70, 80, 90, 95, 99% or any range or value derivable therein.

In further embodiments, the subjects to be treated have not previously been treated by radiation therapy or have been previously treated radiation therapy but then exposed to accidental or terrorist-related radiological event. Fractionated doses are often used in radiation therapy. These large total doses are delivered in small daily amounts over a period of time. Fractionated doses are less effective at inducing ARS than a single dose of the same magnitude.

The subjects to be treated may have one or more of the classic ARS Syndromes, including bone marrow syndrome, gastrointestinal (GI) syndrome, and cardiovascular (CV)/central nervous System (CNS) syndrome.

Bone marrow syndrome (sometimes referred to as hematopoietic syndrome): the full syndrome will usually occur with a dose greater than approximately 0.7 Gy (70 rads) although mild symptoms may occur as low as 0.3 Gy or 30 rads. The survival rate of patients with this syndrome decreases with increasing dose. The primary cause of death is the destruction of the bone marrow, resulting in infection and hemorrhage.

Gastrointestinal (GI) syndrome: the full syndrome will usually occur with a dose greater than approximately 10 Gy (1000 rads) although some symptoms may occur as low as 6 Gy or 600 rads. Survival is extremely unlikely with this syndrome. Destructive and irreparable changes in the GI tract and bone marrow usually cause infection, dehydration, and electrolyte imbalance. Death usually occurs within 2 weeks.

Cardiovascular (CV)/Central Nervous System (CNS) syndrome: the full syndrome will usually occur with a dose greater than approximately 50 Gy (5000 rads) although some symptoms may occur as low as 20 Gy or 2000 rads. Death may occur within 3 days. Death likely is due to collapse of the circulatory system as well as increased pressure in the confining cranial vault as the result of increased fluid content caused by edema, vasculitis, and meningitis.

The subjects having ARS may include one or more of the following stages of ARS. The classic symptoms for the prodromal stage (N-V-D stage) are nausea, vomiting, as well as anorexia and possibly diarrhea (depending on dose), which occur from minutes to days following exposure. The symptoms may last (episodically) for minutes up to several days. Latent stage: In this stage, the patient looks and feels generally healthy for a few hours or even up to a few weeks. In this stage, the symptoms depend on the specific syndrome and last from hours up to several months. Most patients who do not recover will die within several months of exposure. The recovery process lasts from several weeks up to two years.

Radiation in certain aspects includes high-energy electromagnetic waves (x-rays, gamma rays), particles (alpha particles, beta particles, neutrons). The referenced absorbed dose levels may be from beta, gamma, or x radiation. Neutron or proton radiation produces many of the health effects described herein at lower absorbed dose levels. The dose may not be uniform, but a large portion of the body must have received a high level of radiation, for example, more than 0.7 Gy (70 rads). Alpha particles are energetic helium nuclei emitted by some radionuclides with high atomic numbers (eg, plutonium, radium, uranium); they cannot penetrate skin beyond a shallow depth (<0.1 mm).

Beta particles are high-energy electrons that are emitted from the nuclei of unstable atoms (e.g., cesium-137, iodine-131). These particles can penetrate more deeply into skin (1 to 2 cm) and cause both epithelial and subepithelial damage.

Neutrons are electrically neutral particles emitted by a few radionuclides (eg, californium-252) and produced in nuclear fission reactions (eg, in nuclear reactors); their depth of tissue penetration varies from a few millimeters to several tens of centimeters, depending on their energy. They collide with the nuclei of stable atoms, resulting in emission of energetic protons, alpha and beta particles, and gamma radiation.

Gamma radiation and x-rays are electromagnetic radiation (ie, photons) of very short wavelength that can penetrate deeply into tissue (many centimeters). While some photons deposit all their energy in the body, other photons of the same energy may only deposit a fraction of their energy and others may pass completely through the body without interacting.

Because of these characteristics, alpha and beta particles cause the most damage when the radioactive atoms that emit them are within the body (internal contamination) or, in the case of beta-emitters, directly on the body; only tissue in close proximity to the radionuclide is affected. Gamma rays and x-rays can cause damage distant from their source and are typically responsible for acute radiation syndromes (ARS).

Although the dose ranges provided in this document apply to most healthy adult members of the public, a great deal of variability of radiosensitivity among individuals exists, depending upon the age and condition of health of the individual at the time of exposure. Children and infants are especially sensitive.

Conventional units of measurement of radiation include the roentgen, rad, and rem. The roentgen (R) is a unit of exposure measuring the ionizing ability of x-rays or gamma radiation in air. The radiation absorbed dose (rad) is the amount of that radiation energy absorbed per unit of mass. Because biologic damage per rad varies with radiation type (e.g., it is higher for neutrons than for x-rays or gamma radiation), the dose in rad is corrected by a quality factor; the resulting equivalent dose unit is the roentgen equivalent in man (rem). Outside the US and in the scientific literature, SI (International System) units are used, in which the rad is replaced by the gray (Gy) and the rem by the sievert (Sv); 1 Gy=100 rad and 1 Sv=100 rem. The rad and rem (and hence Gy and Sv) are essentially equal (i.e., the quality factor equals 1) when describing x-rays or gamma or beta radiation. The amount (quantity) of radioactivity is expressed in terms of the number of nuclear disintegrations (transformations) per second. The becquerel (Bq) is the SI unit of radioactivity; one Bq is 1 disintegration per second (dps). In the US system, one curie is 37 billion Bq.

In certain aspects, the subjects to be treated may be exposed to radiation that may involve contamination and/or irradiation. Radioactive contamination is the unintended contact with and retention of radioactive material, usually as a dust or liquid. Contamination may be external or internal. External contamination is that on skin or clothing, from which some can fall or be rubbed off, contaminating other people and objects. Internal contamination is unintended radioactive material within the body, which it may enter by ingestion, inhalation, or through breaks in the skin. Once in the body, radioactive material may be transported to various sites (e.g., bone marrow), where it continues to emit radiation until it is removed or decays. Internal contamination is more difficult to remove. Although internal contamination with any radionuclide is possible, historically, most cases in which contamination posed a significant risk to the patient involved a relatively small number of radionuclides, such as phosphorus-32, cobalt-60, strontium-90, cesium-137, iodine-131, iodine-125, radium-226, uranium-235, uranium-238, plutonium-238, plutonium-239, polonium-210, and americium-241.

Irradiation is exposure to radiation but not radioactive material (i.e., no contamination is involved). Radiation exposure can occur without the source of radiation (e.g., radioactive material, x-ray machine) being in contact with the person. When the source of the radiation is removed or turned off, exposure ends. Irradiation can involve the whole body, which, if the dose is high enough, can result in systemic symptoms and radiation syndromes (see Acute radiation syndromes (ARS)), or a small part of the body (eg, from radiation therapy), which can result in local effects. People do not emit radiation (ie, become radioactive) following irradiation.

Diagnosis of radiation exposure is by history of exposure, symptoms and signs, and laboratory testing. The onset, time course, and severity of symptoms can help determine radiation dose and thus also help triage patients relative to their likely consequences. However, some prodromal symptoms (e.g., nausea, vomiting, diarrhea, tremors) are nonspecific, and causes other than radiation should be considered. Many patients without sufficient exposure to cause acute radiation syndromes may present with similar, nonspecific symptoms, particularly after a terrorist attack or reactor accident, when anxiety is high.

After acute radiation exposure, CBC with differential and calculation of absolute lymphocyte count is done and repeated 24, 48, and 72 h after exposure to estimate the initial radiation dose and prognosis (see Table 4: Relationship Between Absolute Lymphocyte Count in the Adult at 48 h, Radiation Dose,* and Prognosis). The relationship between dose and lymphocyte counts can be altered by physical trauma, which can shift lymphocytes from the interstitial spaces into the vasculature, raising the lymphocyte count. This stress-related increase is transient and typically resolves within 24 to 48 h after the physical insult. The CBC is repeated weekly to monitor bone marrow activity and as needed based on the clinical course. Serum amylase level rises in a dose-dependent fashion beginning 24 h after significant radiation exposure, so levels are measured at baseline and daily thereafter.

Other laboratory test are done if feasible:

C-reactive protein (CRP) level: CRP increases with radiation dose; levels show promise to discriminate between minimally and heavily exposed patients.

Blood citrulline level: Decreasing citrulline levels indicate GI damage.

Blood fms-related tyrosine kinase-3 (FLT-3) ligand levels: FLT-3 is a marker for hematopoietic damage.

IL-6: Marker is increased at higher radiation doses.

Quantitative granulocyte colony-stimulating factor (G-CSF) test: Levels are increased at higher radiation doses.

Cytogenetic studies with over dispersion index: These studies are used to evaluate for partial body exposure.

When contamination is suspected, the entire body should be surveyed with a thin window Geiger-Muller probe attached to a survey meter (Geiger counter) to identify the location and extent of external contamination. Additionally, to detect possible internal contamination, the nares, ears, mouth, and wounds are wiped with moistened swabs that are then tested with the counter. Urine, feces, and emesis should also be tested for radioactivity if internal contamination is suspected.

### II. Pharmaceutical Compositions and Routes of Administration

In certain embodiments, there may be provided methods and compositions involving pharmaceutical compositions that comprise biguanide compounds, alone or in combination with a second drug. Biguanide compounds may include, but not be limited to, metformin, phenoformin, buformin, proguanil, Dimethylamine, Trimethylamine, Unsymmetrical dimethylhydrazine, N-Nitrosodimethylamine, Dithiobiuret, Diethylamine, Triethylamine, Diisopropylamine, Dimethylaminopropylamine, Diethylenetriamine, N,N-Diisopropylethylamine, Triisopropylamine, Tris(2-aminoethyl)amine, Mechlorethamine, HN1 (nitrogen mustard), HN3 (nitrogen mustard). Biguanide drugs such as metformin, have been shown to sensitize human lung cancer cells to ionizing radiation (see, e.g., Tsakiridis et al. Abstract 2491, 102nd Meeting American Association for Cancer Research, 2011; Kim et al. Abstract 2869, 102nd Meeting American Association for Cancer Research, 2011).

In particular aspects, the second drug is phosphorothioates compounds. A general description of the class of phosphorothioates compounds and their properties described in this application can be found in Sweeney, 1979 and Giambarresi and Jacobs, 1987, both of which are incorporated by reference. Compounds and designations exemplary of the class of phosphorothioates include S-2-(3-aminopropylamino)ethyl phosphorothioic acid (amifostine, WR-2721), 2-[(aminopropyl)amino] ethanethiol (WR-1065), S-1-(aminoethyl) phosphorothioc acid (WR-638), S-[2-(3-methylaminopropyl)aminoethyl]phosphorothioate acid (WR-3689), S-2-(4-aminobutylamino)ethylphosphorothioic acid (WR-2822), 3-[(2-mercapto ethyl)amino]propionamide p-toluenesulfonate (WR-2529), S-1-(2-hydroxy-3-amino)propyl phosphorothioic acid (WR-77913), 2-[3-(methylamino)propylamino]ethanethiol (WR-255591), S-2-(5-aminopentylamino)ethyl phosphorothioic acid (WR-2823), [2-[(aminopropypamino] ethanethiol]N,N,′-dithiodi-2,1-(ethanediyl)bis-1,3-propanediamine (WR-33278),1-[3-(3-aminopropyl)thiazolidin-2-Y1]-D-gluco-1,2,3,4,5 pentane-pentol dihydrochloride (WR-255709), 3-(3-methylaminopropylamino)propanethiol dihydrochloride (WR-151326), and S-3-(3-methylaminopropylamino)propyl phosphorothioic acid (WR-151327).

In further embodiments, the second drug is a sulfhydryl compound, or a prodrug or salt thereof. The sulfhydryl compound may be any compounds that have a sulfhydryl group. In some embodiments, the therapeutic compound or second drug is a sulfhydryl compound selected from the group consisting of an aminothiol compound such as a thiol form of mifostine, an angiotensin converting enzyme inhibitor, a detoxifying agent, an anti-mucolytic agent, and a combination thereof.

The compounds useful in the methods may be in the form of free acids, free bases, or pharmaceutically acceptable addition salts thereof. Such salts can be readily prepared by treating the compounds with an appropriate acid. Such acids include, by way of example and not limitation, inorganic acids such as hydrohalic acids (hydrochloric, hydrobromic, hydrofluoric, etc.), sulfuric acid, nitric acid, and phosphoric acid, and organic acids such as acetic acid, propanoic acid, 2-hydroxyacetic acid, 2-hydroxypropanoic acid, 2-oxopropanoic acid, propandioic acid, and butandioic acid. Conversely, the salt can be converted into the free base form by treatment with alkali.

Aqueous compositions in some aspects comprise an effective amount of the therapeutic compound, further dispersed in pharmaceutically acceptable carrier or aqueous medium. The phrases “pharmaceutically or pharmacologically acceptable” refer to compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof, and in oils. Under ordinary conditions of storage and use, these preparations contain a preservative to prevent the growth of microorganisms.

The therapeutic compositions may be advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain at least about, at most about, or about 1, 5, 10, 25, 50 mg or up to about 100 mg of human serum albumin per milliliter of phosphate buffered saline. Other pharmaceutically acceptable carriers include aqueous solutions, non-toxic excipients, including salts, preservatives, buffers and the like.

In particular aspects, a form of administration is the use of an auto-injector that can be pre-loaded with a “unit dose” (see below), or calibrated to reliably and/or repeatably deliver a unit dose of therapeutic compounds such as metformin or phosphorothioate. Most autoinjectors are spring-loaded syringes. By design, autoinjectors are easy to use and are intended for self-administration by patients, or administration by untrained personnel. The site of injection depends on the drug loaded, but it typically is administered into the thigh or the buttocks. The injectors were initially designed to overcome the hesitation associated with self-administration of the needle-based drug delivery device. For example, the autoinjector keeps the needle tip shielded prior to injection and also has a passive safety mechanism to prevent accidental firing (injection). Injection depth can be adjustable or fixed and a function for needle shield removal may be incorporated. Just by pressing a button, the syringe needle is automatically inserted and the drug is delivered. Once the injection is completed some auto injectors have visual indication to confirm that the full dose has been delivered. Autoinjectors contain glass syringes, this can make them fragile and contamination can occur. More recently companies have been looking into making autoinjectors syringes out of plastic to prevent this issue. Anapen®, EpiPens®, or the recently introduced Twinject®, are often prescribed to people who are at risk for anaphylaxis. Rebiject® and Rebiject® II autoinjectors are used for Rebif, the drug for interferon β-1a, used to treat Multiple Sclerosis. SureClick® autoinjector delivers a combination product for drugs Enbrel or Aranesp to treat arthritis or anemia, respectively. Any of these technologies could be adapted to deliver the compounds.

Examples of non-aqueous solvents include propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well-known parameters.

The therapeutic compositions may include classic pharmaceutical preparations. Administration of therapeutic compositions will be via any common route so long as the target tissue is available via that route. This includes oral, nasal, buccal, rectal, vaginal or topical. Alternatively, administration will be by orthotopic, intradermal subcutaneous, intramuscular, intraperitoneal or intravenous injection. Such compositions would normally be administered as pharmaceutically acceptable compositions that include physiologically acceptable carriers, buffers or other excipients. Volume of an aerosol is typically between about 0.01 mL and 0.5 mL.

Additional formulations may be suitable for oral administration. “Oral administration” as used herein refers to any form of delivery of an agent or composition thereof to a subject wherein the agent or composition is placed in the mouth of the subject, whether or not the agent or composition is swallowed. Thus, ‘oral administration’ includes buccal and sublingual as well as esophageal administration. Absorption of the agent can occur in any part or parts of the gastrointestinal tract including the mouth, esophagus, stomach, duodenum, ileum and colon. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In one embodiment, the oral formulation can comprise the therapeutic compounds and one or more bulking agents. Suitable bulking agents are any such agent that is compatible with the therapeutic compounds including, for example, lactose, microcrystalline cellulose, and non-reducing sugars, such as mannitol, xylitol, and sorbitol. One example of a suitable oral formulations includes spray-dried therapeutic compounds-containing polymer nanoparticles (e.g., spray-dried poly(lactide-co-glycolide)/amifostine nanoparticles having a mean diameter of between about 150 nm and 450 nm; see, Pamujula, S. et a., J. Pharmacy Pharmacol. 2004, 56, 1119-1125, which is here by incorporated by reference in its entirety). The nanoparticles can contain between about 20 and 50 w/w % therapeutic compounds for example, between about 25% and 50%.

In some embodiments, when the route is topical, the form may be a cream, ointment, salve or spray. Topical formulations may include solvents such as, but not limited to, dimethyl sulfoxide, water, N,N-dimethylformamide, propylene glycol, 2-pyrrolidone, methyl-2-pyrrolidone, and/or N-methylforamide. To enhance skin permeability, if necessary, the skin area to be treated can be pre-treated with dimethylsulfoxide; see Lamperti et al., Radiation Res. 1990, 124, 194-200, which is hereby incorporated by reference in its entirety.

In other embodiments, the therapeutic compositions may be for subcutaneous administration (e.g., injection and/or implantation). For example, implantable forms may be useful for patients which are expected to undergo multiple CT scans over an extended period of time (e.g., one week, two weeks, one month, etc.). In one example, such subcutaneous forms can comprise the therapeutic compounds and a carrier, such as a polymer. The polymers may be suitable for immediate or extended release depending on the intended use. In one example, the therapeutic compounds can be combined with a biodegradable polymer (e.g., polylactide, polyglycolide, and/or a copolymers thereof). In another example, subcutaneous forms can comprise a microencapsulated form of the therapeutic compounds, see, e.g., Srinivasan et al., Int. J. Radiat. Biol. 2002, 78, 535-543, which is hereby incorporated by reference in its entirety. Such microencapsulated forms may comprise the therapeutic compounds and one or more surfactant and other excipients (e.g., lactose, sellulose, cholesterol, and phosphate- and/or stearate-based surfactants).

In a further embodiment, the therapeutic compounds may be administered transdermally through the use of an adhesive patch that is placed on the skin to deliver the therapeutic compounds through the skin and into the bloodstream. An advantage of the transdermal drug delivery route relative to other delivery systems such as oral, topical, or intravenous is that the patch provides a controlled release of the therapeutic compound into the patient, usually through a porous membrane covering a reservoir of the therapeutic compound or through body heat melting thin layers of therapeutic compound embedded in the adhesive. In practicing certain aspects, any suitable transdermal patch system may be used including, without limitation, single-layer drug-in-adhesive, multi-layer drug-in-adhesive, and reservoir.

The therapeutic compositions may optionally further comprise a second protective agent. The second therapeutic agent can be an antioxidant. Examples of suitable antioxidants include, but are not limited to ascorbic acid (vitamin C), glutathione, lipoic acid, uric acid, β-carotene, lycopene, lutein, resveratrol, retinol (vitamin A), α-tocopherol (vitamin E), ubiquinol, selenium, and catalase. In certain embodiments, the second therapeutic agent is vitamin E, selenium or catalase.

An effective amount of the pharmaceutical composition is determined based on the intended goal, such as enhancing or extending the lifespan of a beta cell under hyperglycemic conditions. The term “unit dose” or “dosage” refers to physically discrete units suitable for use in a subject, each unit containing a predetermined quantity of the therapeutic composition calculated to produce the desired responses, discussed above, in association with its administration, i.e., the appropriate route and treatment regimen. The quantity to be administered, both according to number of treatments and unit dose, depends on the protection desired. An effective dose is understood to refer to an amount necessary to achieve a particular effect, for example, an increased antioxidant capability of a cell. In the practice in certain embodiments, it is contemplated that doses in the range from 10 mg/kg to 200 mg/kg can affect the protective capability of these compounds. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM. In other embodiments, the dose can provide the following blood level of the compound that results from a therapeutic compound being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic compound that is administered to a subject is metabolized in the body to a metabolized therapeutic compound, in which case the blood levels may refer to the amount of that compound. Alternatively, to the extent the therapeutic compound is not metabolized by a subject, the blood levels discussed herein may refer to the unmetabolized therapeutic compound.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware of this invention that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### III. Kits and Other Apparatuses or Systems

Certain aspects also encompass kits, medical devices or apparatuses, or systems for performing the methods described herein. Such embodiments can be prepared from readily available materials and reagents. For example, such embodiments can comprise any one or more of the following materials: therapeutic compounds and pharmaceutically suitable carriers. In a particular embodiment, these kits include the needed apparatus for administration, like syringes. Instructions for administration can also be included in the kits.

The components may be packaged either in aqueous media or in lyophilized form. The container means will generally include at least one vial, test tube, flask, bottle, syringe or other container means, into which a component may be placed, and preferably, suitably aliquotted. Where there is more than one component (labeling reagent and label may be packaged together), the kit, device or apparatus, or system also will generally contain a second, third or other additional container into which the additional components may be separately placed. However, various combinations of components may be comprised in a vial. They also will typically include a means for containing the therapeutic compounds, and any other reagent containers in close confinement for commercial sale. Such containers may include injection or blow molded plastic containers into which the desired vials are retained.

When the components are provided in one and/or more liquid solutions, the liquid solution is an aqueous solution, with a sterile aqueous solution being one embodiment.

However, the components may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the pharmaceutical formulations are placed, preferably, suitably allocated. The kits, devices/apparatuses, or systems may also comprise a second container means for containing a sterile, pharmaceutically acceptable buffer and/or other diluent.

They may include a means for containing the vials in close confinement for commercial sale, such as, e.g., injection and/or blow-molded plastic containers into which the desired vials are retained.

Such generally will comprise, in suitable means, distinct containers for each individual reagent or solution.

These embodiments may also include instructions for employing the components as well the use of any other reagent not included in the kit. Instructions may include variations that can be implemented.

In particular aspects, the kit may be a transdermal patch. Transdermal patches for use in certain aspects can be of any design known in the art, including specialized patches for iontophoretic delivery or in conjunction with small electric currents (electroporation), ultrasound or microneedle technology to assist delivery across the skin. Non-limiting examples of suitable patches include reservoir, matrix, multi-layer, drug- in-adhesive, or any type of patch technology known to the art, with or without a rate-limiting membrane to control diffusion of the therapeutic compound(s).

Transdermal patches for a delivery of more than one drug in the same dosage form can be constructed with a single reservoir, matrix or adhesive which contains both drugs, or if biostability or other compatibility problems exist, can be constructed with two separate reservoirs, adhesives or matrices, one for each compound. Transdermal administration of various pharmaceutical compositions has been described previously. In adhesive patches, a drug may be dissolved or suspended directly in the adhesive which contacts the skin. Reservoir transdermal systems include a liquid or semi-liquid compartment containing a drug suspension or solution, separated from the skin by a semi-permeable membrane. In matrix transdermal systems, a drug may be contained within a solid or semi-solid matrix which contacts the skin of the user and is surrounded at the perimeter by an adhesive. These different transdermal systems are described in, for example, U.S. Pat. Nos. 4,751,087; 5,372,819; 5,405,317; 6,312,715; 6,322,532, the disclosures of which are hereby incorporated by reference. Exemplary suitable transdermal technologies which are compatible include those used in D-TRANS™, E-TRANS™, MICROFLUX™, LATITUDE™, LATITUDE™ DUO, CLIMARA PRO™, and any other transdermal delivery systems known in the art.

Combination or concomitant therapy involving a transdei many administered a drug such as metformin can minimize the potential side effects associated with drug-drug interactions by reducing the peak plasma concentration of metformin, whether the combination treatment is administered in one dosage form or more than one. Combination therapy or administration in combination generally refers to administration of two or more pharmaceutical active or therapeutic compounds in a single dosage form. Concomitant administration or therapy refers to administration of two or more pharmaceutical active or therapeutic compounds at the same time or in such close proximity in time such that therapeutic levels of each compound exist in the patient at the same or overlapping times. Therefore, the term concomitant administration may encompass combined or combination administration/therapy.

Transdermal patches may comprise one or more drug reservoir compartments or layers. One or more drugs in the form of a viscous liquid is contained in a compartment or layer. Thus, the terms “drug reservoir compartment” and “drug reservoir layer” are used interchangeably throughout the specification and claims and refer to that part of the laminated structure of the patch which comprises or holds the drug formulation. For example, the number of drug reservoir compartments or layers may be determined by the desired release characteristics. The concentration of the active agent in the different compartments or layers may be varied and the thickness of the different compartments or layers need not be the same.

Additionally, the drug reservoir compartment or layer may comprise or hold one or more active agents so as to achieve a desired therapeutic effect. For example, the drug reservoir compartments or layers of the present invention are thin, flexible, and conformable to provide intimate contact with a body skin, and are able to release drugs from the reservoir at rates sufficient to achieve therapeutically effective transdermal fluxes of the drugs. Materials that may be used for drug reservoir compartment are polyurethanes, polyolefins such as polyethylene and polypropylene, silicone, ethylene-ethacrylate copolymer, ethylene-vinyl acetate copolymer, ethylene-vinyl methylacetate copolymer, polytetrafluoroethylene (“Teflon”), polycarbonate, polyvinylidene difmoride (PVDF), polycarbonate, polyvinylidene difluoride (PVDF), polysulfones, and the like.

Transdermal patches may comprise one or more rate or non-rate controlling layers, which are usually microporous membranes. The rate or non-rate controlling layers comprise biopolymers and/or synthetic polymers. The rate or non-rate controlling layers are devoid of an active agent. Representative materials useful for forming rate or non-rate controlling layers include, but are not limited to, polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, cellulose acetate and cellulose nitrate, polytetrafluoroethylene (“Teflon”), polycarbonate, polyvinylidene difluoride (PVDF), polysulfones, and the like.

The various layers may contact each other by any method known in the art. One such method is to place layers adjacent to each other and apply pressure to the outer sides of the layers to force the layers together. Another method is to coat the surface of each of the layers to be contacted with a solvent, such as water, before placing the layers together. In this way, a thin portion of each surface will become soluble and/or swollen thereby producing adhesion upon contact. Another method is to use a known adhesive on one or more of the contacting surfaces. Preferably, the adhesive is one that will not interfere with the delivery of the active agent from the drug reservoir layer.

In some embodiments, transdermal patches may be used to administer metformin or other therapeutic compounds described herein, which is present in one or more drug reservoir compartments or layers. The drug reservoir layer may itself have adhesive properties, or the patch may further comprise an adhesive layer attached to the drug reservoir layer. The patch may further comprise a backing layer. For example, a backing layer functions as the primary structural element of a transdermal patch and provides flexibility and, preferably, occlusivity. The material used for the backing layer should be inert and incapable of absorbing an active agent or any component of the formulation contained within the drug reservoir layer. The backing layer may comprise a flexible and/or elastomeric material that serves as a protective covering to prevent loss of the active agent via transmission through the upper surface of the patch, and may impart a degree of occlusivity to the patch, such that the area of the body surface covered by the patch becomes hydrated during use. The backing layer may also prevent dehydration of the drug reservoir layer.

The material used for the backing layer should permit the patch to follow the contours of the skin and be worn comfortably on areas of skin such as at joints or other points of flexure, that are normally subjected to mechanical strain with little or no likelihood of the patch disengaging from the skin due to differences in the flexibility or resiliency of the skin and the patch. Examples of materials useful for the backing layer are polyesters, polyolefins including monolayers or coextruded multilayers, polyethylene, polypropylene, vinyliden chloride/vinyl chloride copolymer, ethylene/vinyl acetate copolymer, polyurethanes, polyether amides, and the like. The occlusive backing layer may be covered by an adhesive layer to allow sticking the patch on to the skin

During storage and prior to use, transdermal patches may include a release liner. Immediately prior to use, this layer is removed so that the patch may be affixed to the skin. The release liner should be made from a drug impermeable material, and is a disposable element, which serves only to protect the patch prior to application.

One or more active drugs, agents and/or analogs thereof can be administered topically or transdermally. When given by this route, the appropriate dosage form may be a cream, ointment, or patch. Because the amount that can be delivered by a patch may be limited, two or more patches may be used.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Cells in Culture. Mouse embryo fibroblasts (MEF) were isolated from 14-16 day old pregnant female C57BL/6 WT mice following a method described in detail elsewhere (Grdina, et al., 2013). Mice were euthanized, and the uterus was removed and placed in a culture dish containing sterile PBS. Organs, tail, limbs and head were removed for genotyping. Embryos were placed in PBS with 0.25% trypsin (Invitrogen Life Technologies) and finely minced with scissors. Minced tissues were incubated for 15 min at 37° C. and pipetted to dissociate the tissue. This process was repeated two to three times after which supernatants were collected and centrifuged. Cells were re-suspended in culture medium containing (1:1) Dulbecco's Modified Eagle's medium (DMEM):F12 (Invitrogen Life Technologies), 10% fetal bovine serum (FBS, Atlanta Biologicals, Lawrenceville, Ga.), 100 units/ml penicillin and 100 mg/ml streptomycin (Invitrogen Life Technologies), and plated in 100-mm diameter dishes at a density of 106 cells/dish. MEF were then transformed with c-myc and H-Ras according to a method described in detail elsewhere to immortalize them (Grdina, et al., 2013). SA-NH cells derived from an SA-NH murine sarcoma tumor and adapted for in vitro growth were grown and cultured as described in detail elsewhere (Murley, et al., 2002). Human microvascular endothelial cells (HMEC) from human dermis immortalized with SV40 were maintained in endothelial basal medium MCDB131 (Gibco/BRL, Grand Island, N.Y.) according to culture conditions described elsewhere (Murley, et al., 2006). All cell cultures were maintained at 37° C. in a humidified environment containing 5% CO₂ and were grown to exponential phase for irradiation and drug treatment.

In Vitro Cell Survival Assay. All cells were irradiated with 4 Gy x-rays using a Philips X-ray generator operating at 250 kVp and 15 mA at a dose rate of 0.368 Gy/min (Grdina, et al., 2013). Unirradiated cells served as controls. Immediately following irradiation, cells were counted, diluted, and known numbers seeded into 100-mm tissue culture dishes to allow the development of 50 to 150 colonies per dish. Colonies were stained with 20% crystal violet and scored 12 days after plating. Five dishes per experimental point were used and experiments were repeated three times (Grdina, et al., 2013; Murley, et al., 2002; Murley, et al., 2006).

Mice. Female C3H mice between 8 and 11 weeks old were supplied by Harlan Laboratories (Indianapolis, Ind.) and allowed a minimum of 1 week to acclimate to the University of Chicago animal facility. They were provided standard laboratory rodent chow and clean water ad libitum. Mice were housed five to a plastic cage under standard conditions (12 h light and 12 h dark in humidified 48% air and a constant temperature of 22° C.). The care and treatment of animals was in accordance with institutional guidelines and adherence to the NIH Guide for the Care and Use of Laboratory Animals.

Drugs. Metformin (1, 1-Dimethylbiguanide hydrochloride from Sigma-Aldrich) was dissolved in PBS (phosphate buffered saline from Gibco Company) and filter sterilized. Concentrations of Metformin for studies with cells in culture were evaluated in the range from 1 mM to 20 mM. No cyto-toxicity was evidenced at any of these concentrations. A 5 mM concentration of Metformin was chosen for routine use in all subsequent in vitro studies. WR1065 (Drug Synthesis and Chemistry Branch, Division of Cancer Treatment, National Cancer Institute, Bethesda, Md.) was used either alone prior to irradiation as a positive control at its maximum cytoprotective concentration of 4 mM (Murley, et al., 2004) or in combination with metformin at select times following irradiation.

For in vivo studies, mice were injected i.p. with a final volume of 0.2 ml for all single and drug combinations used. Metformin was diluted to result in a final concentration of 250 mg/kg body weight. Captopril was injected at a final concentration of 200 mg/kg body weight, MESNA was injected at a final concentration of 300 mg/kg body weight, and NAC was injected at a concentration of 400 mg/kg body weight. These concentrations were chosen because they were non-toxic. Amifostine (Drug Synthesis and Chemistry Branch, Division of Cancer Treatment, National Cancer Institute) was administered i.p. 30 min prior to irradiation to achieve a final dose of 400 mg/kg body weight, a concentration known to afford maximum radioprotection in the C3H mouse model (Grdina, et al., 2000).

In Vivo Irradiation and drug treatments. Mice at 8-11 weeks of age were placed in a cylindrical clear-plastic holder and exposed to r-rays at room temperature using an X-ray generator (RT250 manufactured by Philips) operated at 250 kVp and 15 mA at a dose rate of 1.33 Gy/min. Based on early studies, a dose of 7 Gy x-rays produced on average 10 nodules per spleen using the endogenous spleen colony assay of Till and McCulloch (Till, et al., 1963). All drug preparations were made just prior to their use. Amifostine was injected 30 mM prior to irradiation to serve as a positive control while metformin alone or in combination with each of the sulfhydryl containing drugs was injected 24 h following irradiation.

Spleen colony assay. The classical endogenous spleen colony was used to assess radioprotector efficacy (Till, et al., 1963). Mice were euthanized 13 days after irradiation and drug treatment and spleens were removed and placed in Bouin's solution (Sigma-Aldrich) and allowed to soak for a minimum of 30 minutes before being examined for nodules appearing on the surface of the spleens.

Statistical Analysis. Means and standard errors were calculated for all data points from at least three independent experiments. Pairwise comparisons of cell survival and apoptosis frequencies between each of the experimental conditions were performed using a Student's two-tailed t test (SigmaPlot software 11.0, SPSS, Chicago, Ill.).

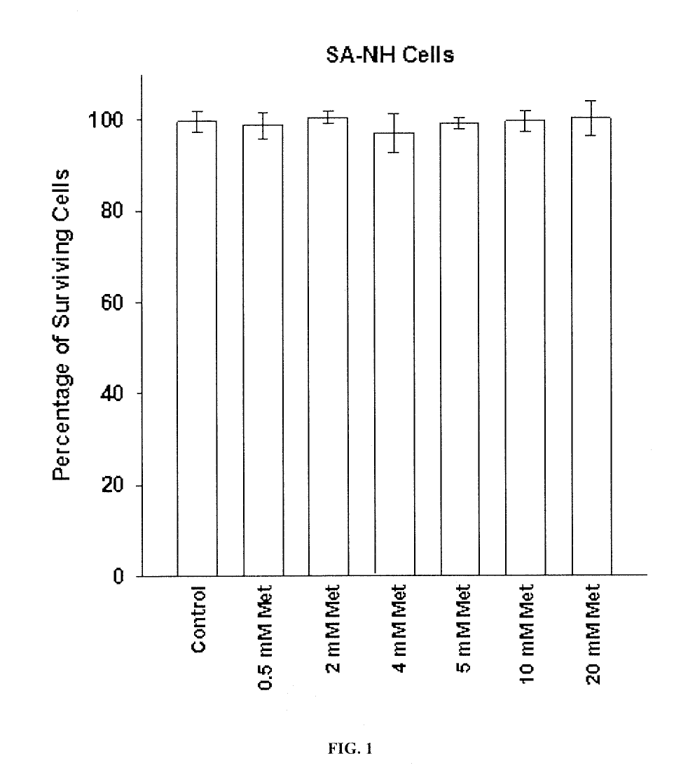

In Vitro Toxicity Assessment of Metformin. Presented in FIG. 1 is a toxicity assessment for a one h exposure of metformin on SA-NH murine tumor cells over a 0.5 to 20 mM concentration range. Metformin exhibited no cyto-toxicity throughout this dose range. A concentration of 5 mM was chosen for all subsequent experiments.

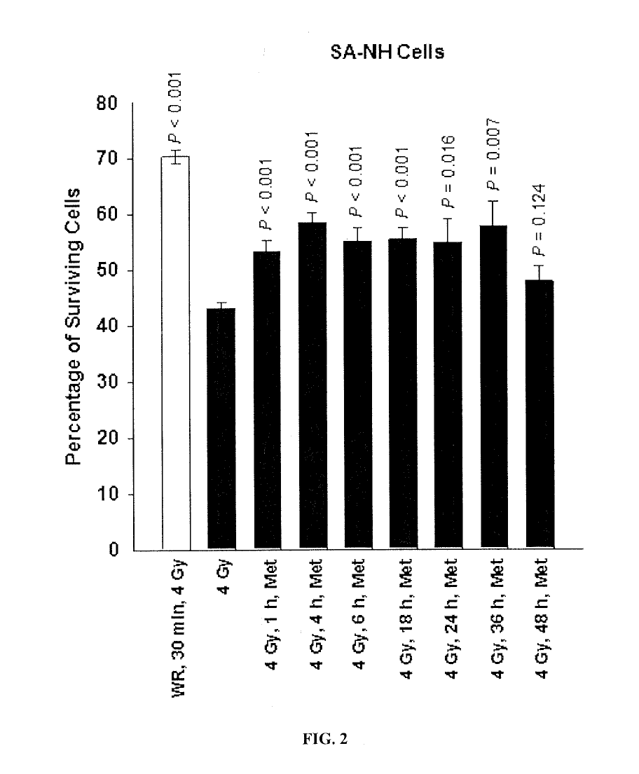

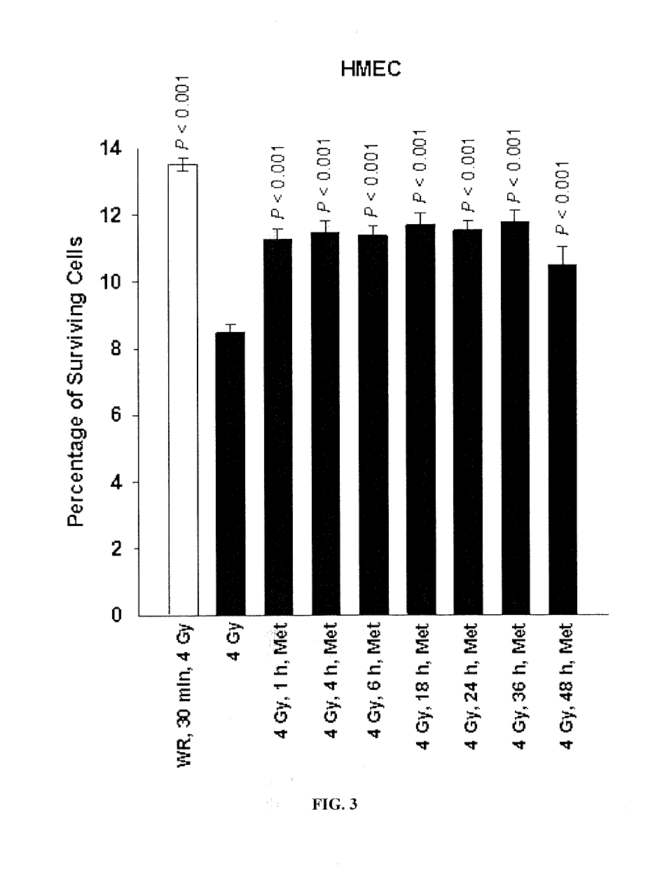

In Vitro Time Course of Metformin Effectiveness Following Radiation Exposure. SA-NH, HMEC, and MEF cells were each exposed to 5 mM of metformin for 1 hour at time increments of 1, 4, 6, 18, 24, 36, or 48 h following exposure to 4 Gy. As demonstrated in FIGS. 2, 3, and 4, metformin was effective in protecting against radiation-induced cell killing of SA-NH by a protection ratio range of 1.2 to 1.3, HMEC a range of 1.3-1.5, and MEF cells a range of 1.4 to 1.6 when administered at times ranging from 1 to 24 h following irradiation. Protection was extended in SA-NH by a factor of 1.3 and HMEC by a factor of 1.5, when metformin was added 36 h later while only HMEC exhibited elevated radiation resistance, a protection ratio of 1.3, if metformin was added 48 h following irradiation. While each of these elevated survival levels reached significance levels of ρ≦0.007 as compared to cells only exposed to 4 Gy, none of the metformin only treatments afforded the level of protection observed if SA-NH, HMEC, or MEF were treated with WR1065 30 min prior to irradiation, e.g., protection ratios of 1.6, 1.8, and 1.9, respectfully.

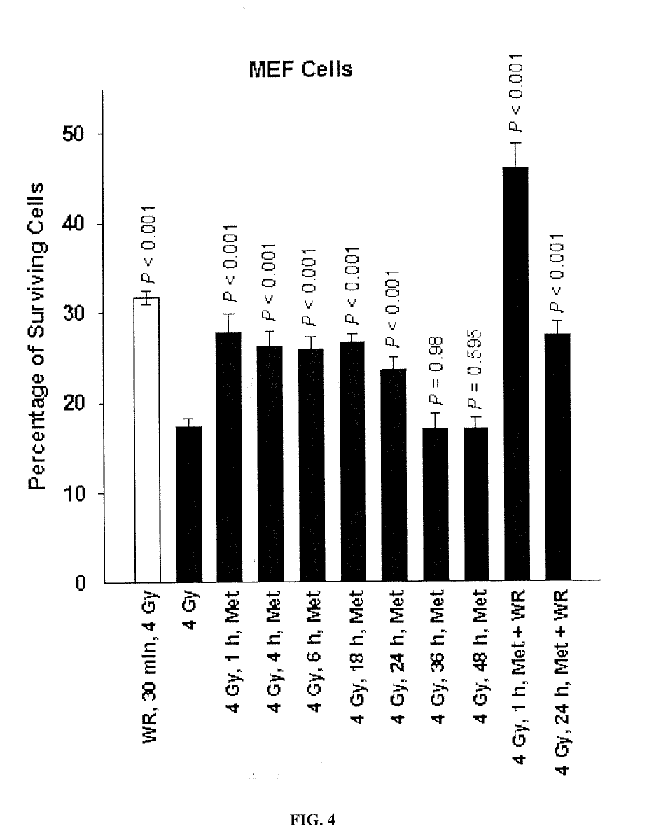

The combination of metformin and WR1065 was evaluated following irradiation only in MEF cells. If this combination of drugs was added 1 h following irradiation, cell survival was significantly enhanced by a protection ratio of 2.7 (see FIG. 4). This combined drug enhancement in protective effectiveness over WR1065 pre-irradiation treatment alone was not observed, however, if the combination of drugs was added 24 h later, e.g., protection ratio of 1.6.

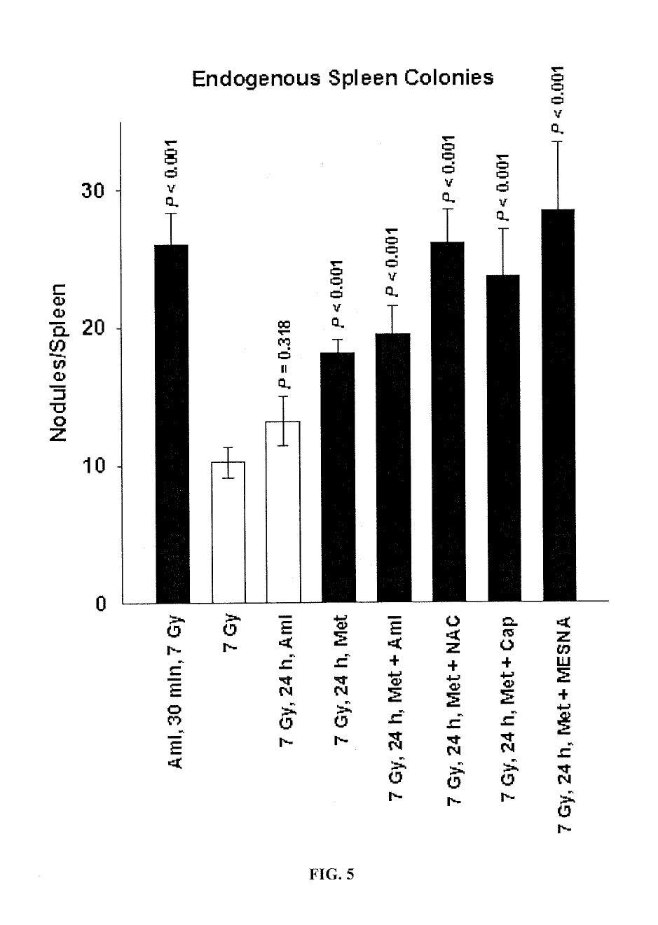

In Vivo Assessment of Metformin Alone or in Combination as a Radiation Mitigator. To assess the efficacy of metformin as a mitigator when used alone or in combination with select FDA approved sulfhydryl containing drugs in protecting against radiation-induced cytotoxicity, a classical in vivo model system for normal tissue toxicity was used. Current regulations at The University of Chicago preclude the use of classical LD50 or LD70 assays to evaluate drugs for their radioprotective effectiveness. For this reason we chose the well characterized endogenous spleen colony assay first described by Drs. Till and McCulloch in 1963 as a direct measurement of the radiation sensitivity of mouse splenocytes (Till, et al., 1963). C3H mice 8 to 11 weeks of age were exposed to a 7 Gy whole body dose of ionizing radiation. Amifostine administered 30 min prior to or 24 h following irradiation was used as a positive control for radioprotector effectiveness. Metformin alone or in combination with amifostine, NAC, Captopril, or MESNA was administered 24 h following irradiation and the data are presented in FIG. 5. A dose of 7 Gy resulted in an average of 10 regenerating nodules on the surface of the spleens. Amifostine administered before irradiation significantly protected the spleens as evidenced by an average of 26 nodules per spleen and a protection ratio of 2.6. Amifostine administered 24 h following irradiation afforded no significant elevation in protection (ρ=0.318). Metformin alone administered 24 h following irradiation significantly protected irradiated animals (ρ<0.001). The average number of spleen nodules was 18 giving rise to a 1.8 protection ratio. The combination of metformin with amifostine, NAC, Captopril, or MESNA resulted in greater elevations in protection ratios of 2.0, 2.6, 2.4, and 2.8, respectfully, approaching or exceeding that observed for amifostine only when administered 30 min prior to irradiation, e.g. protection ratio of 2.6.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Algire, et al., Cancer Prey Res. 5(4):536-543, 2012.

Coleman, et al., Science. 304:693-694, 2004.

Evans, et al., Brit Med J. 330:1304-1305, 2005.

Gilmore & Shadduck, Blood. 85(10):2731-4, 1995.

Goodarzi & Bryer-Ash, Diabetes Obes Metab. 7(6):654-65, 2005.

Grdina, et al., Drug Metabol Drug Inter. 16:237-279, 2000.

Grdina, et al., Radiat Res. 179:115-124, 2013.

Halicka, et al., Aging. 3(10):1-11, 2011.

Kinnamon, et al., Exp Biol Med. 164(3):370-3, 1980.

Martin-Castillo, et al., Cell Cycle, 9(6):1057-1064, 2010.

Muaddi, et al., Radiotherapy and Oncol. 108:446-450, 2013.

Murley, et al., Radiat Res. 162:536-546, 2004.

Murley, et al., Free Rad Biol Med. 40:1004-1016, 2006.

Murley, et al., Radiat Res. 158:101-109, 2002.

Na, et al., Mech Ageing Dev. 134:381-390, 2013.

Patchen, et al., Int J Radial Oncol Biol Phys. 18(5):1069-75, 1990.

Ross & Peake, Br J Cancer. 53(Suppl VII):126-9, 1986.

Skinner, et al., Cancer Med. 2(1):99-107, 2013.

Song, et al., Sci Rep. 2:362 Epub, 2012.

Spiller & Sawyer, Am J Health Syst Pharm. 63(10):929-38, 2006.

Stone, et al., Radiat Res. 162:711-728, 2004.

Storozhuk, et al., Br J Cancer. 108(10):2021-2032, 2013.

Taubes, Science. 335:29, 2012.

Till and McCulloch, Radiat Res. 18:96-105, 1963.

Tosca, et al., Reproduction. 139:409-418, 2010.

Weiss and Landauer, Int J Radiat Biol. 85(7):539-573, 2009.

Zannella, et al., Clin Cancer Res. Oct. 18, Epub, 2013.

1. A method of treating a subject exposed to radiation, comprising administering to the subject an effective amount of a pharmaceutical composition comprising a biguanide compound only after the subject has been exposed to radiation.

2. The method of claim 1, wherein the subject has been exposed to a lethal level of radiation or a radiation suspected of being at a lethal level.

3. The method of claim 1, wherein the biguanide compound is administered at a dosage of 1 to 500 mg/kg weight.

4. The method of claim 1, wherein the biguanide compound is metformin.

5. The method of claim 1, wherein the pharmaceutical composition further comprises a second drug, or wherein the method further comprises administering a separate pharmaceutical composition comprising a second drug.

6. The method of claim 5, wherein the second drug is phosphorothioate compound or a metabolite thereof, a sulfhydryl compound, or a prodrug or salt thereof.

7. (canceled)

8. The method of claim 5, wherein the second drug is amifostine (2-(3-aminopropylamino)ethylsulfanyl phosphonic acid) or 2-[(aminopropyl) amino] ethanethiol (WR1065), or a prodrug or salt thereof.

9. The method of claim 5, wherein the second drug is a sulfhydryl compound that is aminothiol compound, an angiotensin converting enzyme inhibitor, a detoxifying agent, an anti-mucolytic agent, or a combination thereof.

10-13. (canceled)

14. The method of claim 1, wherein the pharmaceutical composition is administered subcutaneously, intravenously, topically, transdermally, by inhalation, or orally.

15-16. (canceled)

17. The method of claim 1, wherein the pharmaceutical composition is administered immediately after the subject is first exposed to radiation.

18. The method of claim 1, wherein the pharmaceutical composition is administered at least about 4 hours after the subject is first exposed to radiation.

19-20. (canceled)

21. The method of claim 1, wherein the pharmaceutical composition is administered at least about 24 hours after the subject is first exposed to radiation.

22. (canceled)

23. The method of claim 1, wherein the pharmaceutical composition is administered at least about 48 hours after the subject is first exposed to radiation.

24-25. (canceled)

26. The method of claim 1, wherein the pharmaceutical composition is administered to the subject after the subject is tested for the level of radiation exposure.

27. The method of claim 1, further comprising monitoring the subject for radiation-induced damage.

28. (canceled)

29. The method of claim 1, further comprising administering multiple doses of the pharmaceutical composition comprising a biguanide compound.

30-32. (canceled)

33. A method of treating a subject exposed to a radiological incident, comprising administering to the subject an effective amount of a pharmaceutical composition comprising a biguanide compound, wherein the radiological incident comprises a lethal level of radiation or a radiation suspected of being at a lethal level.

34-35. (canceled)

36. A kit comprising one or more doses of: a first pharmaceutical composition comprising metformin or a prodrug or salt thereof; and a second pharmaceutical composition comprising a sulfhydryl compound, wherein the first and second pharmaceutical composition are individually packaged.

37-40. (canceled)

41. The method of claim 1, wherein the subject was exposed in a lethal level of radiation or a radiation suspected of being at a lethal level.

42. The method of claim 1, wherein the subject was determined to exhibit the symptoms of acute radiation syndrome or radiation-induced cytotoxicity.

43. The method of claim 1, wherein the radiation is ionizing radiation.
